(19) United States
(12) Patent Application Publication
(10) Pub. No.: US 2020/0066413 A1
(43) Pub. Date: Feb. 27, 2020

(54) MEDICAL PROCEDURE BASED A/R AND V/R EXPERIENCE BETWEEN PHYSICIAN AND PATIENT/PATIENT'S FAMILY

(71) Applicant: INTERNATIONAL BUSINESS MACHINES CORPORATION, Armonk, NY (US)

(72) Inventors: Matthew E. Broomhall, Goffstown, NH (US); Paul R. Bastide, Boxford, MA (US); Lin Sun, Cary, NC (US); Liam S. Harpur, Dublin (IE)

(21) Appl. No.: 16/113,993

(22) Filed: Aug. 27, 2018

(51) Int. Cl. G16H 80/00; G06F 3/0481; G06T 19/00; A61B 5/16

(52) U.S. Cl. CPC G16H 80/00; G06F 3/04815; A61B 5/0816; A61B 5/165; G06T 19/006

(57) ABSTRACT

Embodiments of the present disclosure relate to improving the experience between a patient and a healthcare provider using augmented or virtual reality to supplement a medical communication interaction, such as a pre-operative check-up. In various embodiments of the invention, a method includes measuring a first physiological property of a patient during a communication interaction with a healthcare provider, where the first physiological property includes natural language communicated by the patient, eye movement, breathing rate, and body position. The method includes determining whether the first physiological property is indicative of a negative interaction between the healthcare provider and the patient. If the communication interaction is negative, an indication is provided to the healthcare provider that the communication interaction is negative. An artificial reality display is provided to supplement the communication interaction by demonstrating a medical procedure.

### BACKGROUND

Embodiments of the present disclosure relate to improving the healthcare experience of patients, and more specifically, to improving the experience between a patient and a healthcare provider using augmented or virtual reality to supplement a medical communication interaction. Patients facing surgery or other medical procedures may lack the ability to express their thoughts or ask questions during important interactions with their healthcare provider (e.g., during a pre-operative check-up). In many cases, fear, anxiety, embarrassment, language barriers, and/or cultural differences may contribute to the patient's difficulties in expressing their thoughts or questions during these interactions. The healthcare provider may explain to the patient information regarding their condition and treatment plan/options, but may be unaware that the patient is understanding very little due to any of the above factors.

### BRIEF SUMMARY

According to embodiments of the present disclosure, systems, methods of and computer program products for improving the experience between patients and healthcare providers are provided. In one or more embodiments of the invention, a method includes measuring a first physiological property of a patient during a communication interaction with a healthcare provider, where the first physiological property selected from the group consisting of: natural language communicated by the patient, eye movement, breathing rate, and body position. The method further includes determining whether the first physiological property is indicative of a negative interaction between the healthcare provider and the patient. When the first physiological property meets a threshold value indicative of a negative interaction, the method includes providing an indication to the healthcare provider that the communication interaction is negative. The method further includes providing an artificial reality display to supplement the communication interaction. The artificial reality display includes an augmented reality (AR) system or a virtual reality (VR) system and demonstrates a medical procedure to the patient.

In one or more embodiments of the invention, a system includes a computing node having a computer readable storage medium having program instructions embodied therewith, the program instructions executable by a processor of the computing node to cause the processor to perform a method including measuring a first physiological property of a patient during a communication interaction with a healthcare provider, where the first physiological property includes natural language communicated by the patient, eye movement, breathing rate, and body position. The method further includes determining whether the first physiological property is indicative of a negative interaction between the healthcare provider and the patient. When the first physiological property meets a threshold value indicative of a negative interaction, the method includes providing an indication to the healthcare provider that the communication interaction is negative. The method further includes generating, for display on an artificial reality display, a medical procedure to the patient, the artificial reality display supplementing the communication interaction and comprising an augmented reality (AR) system or a virtual reality (VR) system.

In one or more embodiments of the invention, a computer program product is provided for improving the experience between a patient and a healthcare provider. The computer program product includes a computer readable storage medium having program instructions embodied therewith, the program instructions executable by a processor to cause the processor to perform a method including measuring a first physiological property of a patient during a communication interaction with a healthcare provider, where the first physiological property includes natural language communicated by the patient, eye movement, breathing rate, and body position. The method further includes determining whether the first physiological property is indicative of a negative interaction between the healthcare provider and the patient. When the first physiological property meets a threshold value indicative of a negative interaction, the method includes providing an indication to the healthcare provider that the communication interaction is negative. The method further includes generating, for display on an artificial reality display, a medical procedure to the patient, the artificial reality display supplementing the communication interaction and comprising an augmented reality (AR) system or a virtual reality (VR) system.

### BRIEF DESCRIPTION OF THE SEVERAL VIEWS OF THE DRAWINGS

FIG. 1 illustrates an exemplary scenario of a healthcare provider interacting with a patient and utilizing the system of the present invention.

FIG. 2 illustrates an exemplary scenario of a healthcare provider interacting with a patient while supplementing the interaction with AR/VR.

FIG. 3 illustrates an exemplary AR environment where bones are simulated from patient medical images.

FIG. 4 illustrates a flow chart illustrating an exemplary method for improving the experience between patients and healthcare providers.

FIG. 5 depicts an exemplary computing node according to embodiments of the present disclosure.

### DETAILED DESCRIPTION

In the clinical practice of medicine, healthcare providers (e.g., doctors, nurses, physical therapists, or other medical professionals) traditionally conducts a series of consultations and/or examinations for patients who are planning to have a surgical procedure performed. During these consultations and/or examinations, the healthcare provider(s) may explain to the patient information regarding the patient's condition, pre-operative preparation steps, the surgical procedure, and post-operative expectations. In many cases, because of emotional factors such as fear, anxiety, anger, and/or embarrassment and other external factors such as language barriers and/or cultural differences, patients may find difficulties in expressing their thoughts or questions during these interactions with their healthcare provider(s). The healthcare provider(s) may explain to the patient information regarding their condition and treatment plan/options, but may be unaware that the patient is understanding very little due to any of the above factors. This may not be the result of poor explanation on the healthcare provider's part, but rather on the mental state of the patient affecting their ability to process information regarding their treatment.

The systems, methods, and computer program products of the present invention allow healthcare providers to gain insight into whether or not their patients are fully understanding in-person communications/interactions regarding their healthcare. In particular, the systems, methods, and computer program products of the present invention may constantly monitor in-person communication interactions between the healthcare provider and the patient, through an analysis of a multitude of behavioral and physiological factors, to provide a discrete notification to the healthcare provider if it is determined that the patient is exhibiting signs of not understanding or confusion. Non-limiting examples of a healthcare communication interaction between a healthcare provider and a patient may include a pre-operative check-up or evaluation or an annual physical wellness exam. During this communication interaction, the healthcare provider may explain to the patient information such as, for example, a medical diagnostic test, results of a medical diagnostic test, a surgical procedure, pre-operative preparation instructions, post-operative recovery process, or other healthcare-related information. These types of discussions can be stressful for patients, and this stress may make it difficult for the patient to adequately express their thoughts or questions to the healthcare provider.

The system of the present invention includes at least one recording device to record the communication interaction between the healthcare provider and the patient. The recording device may include a camera, such as, for example, a digital camera, a stereoscopic camera, a plenoptic camera, a thermal camera, a fish-eye camera or any other suitable recording device as is known in the art. The recording device may also include one or more microphones, either separately or integrated into the device. The camera(s) may be used to record images and/or video of the interaction while the microphone may be used to record audio data associated with the interaction between the health care provider and the patient. Audio data may include, for example, natural language from either or both of the healthcare provider and the patient.

The system of the present invention may process the recorded images, video, and or audio data using computer vision techniques, audio processing techniques, and/or natural language processing techniques as is known in the art. From the recorded images, video, and/or audio data, the system may measure one or more physiological or behavioral factors/properties of one or both of the patient and the healthcare provider during the interaction. For example, the system may measure the natural language of the patient or the healthcare provider (e.g., intonation, sentiment, complexity of vocabulary, and/or length of time each individual is talking). As another example, the system may measure physiological factors/properties such as eye movement or blinking rate, breathing rate, and/or body position. For example, body position may include the posture or limb motion of the patient and/or the healthcare provider. Behavioral factors/properties may also be measured. For example, the system may measure pathing of the patient, such as if the patient is pacing in a room, where excessive pacing may indicate that the patient is stressed or anxious.

The system of the present invention may determine whether one or more of the above-listed physiological or behavioral factors/properties is indicative of a negative interaction. Generally, a negative interaction is where the patient is not able to fully express themselves to their healthcare provider or understand their communications with their healthcare provider due to certain circumstances (e.g., emotional or communication-related). A negative interaction may occur, for example, when a patient is stressed or anxious about an upcoming surgical procedure and is not fully understanding a discussion with their healthcare provider about the surgical procedure. In various embodiments, certain behaviors or physiological properties may be associated with negative interactions, such as, for example, lack of eye contact, too much eye contact, too much tactile interaction, too little tactile interaction, hyperventilation, body position anxiety, hiding a body part, not responding when engaged by the healthcare provider, and/or responding to a different topic.

In various embodiments, the system of the present invention may determine using audio processing of recorded audio data (and/or computer vision processing of the motion of the patient's mouth) that the patient is not asking enough questions and thus determine that the interaction is negative. In various embodiments, the system of the present invention may determine that the patient is asking a lot of questions because they are anxious and thus determine that the interaction is negative. In various embodiments, the system of the present invention may determine through computer vision that the patient's posture is indicative of fear or anxiousness and thus determine that the interaction is negative.

Once the system determines that the interaction is negative, the system may provide a discrete indication to the healthcare provider that the communication interaction is negative. For example, the healthcare provider may have an electronic accessory, such as smart glasses, a smart watch, or a mobile phone/device to which the system can “push” a notification of a negative communication interaction to the healthcare provider. In various embodiments, the notification may be a binary indication such as a light or a symbol (e.g., an emoji, an up/down arrow, a thumbs up/down). In various embodiments, the indicator may be text (e.g. a text message), a numerical scale (e.g., 1 to 10), a sliding scale, or a color bar scale (e.g., green/yellow/red).

In various embodiments, the system of the present invention may determine the quality of interactions between several individuals at the same time (e.g., family members of the patient). In this case, the system may provide one or more notifications to the healthcare provider that members other than the patient are displaying behavioral or physiological patterns indicative of a negative interaction.

Once a negative communication interaction is determined between the healthcare provider and the patient, the system of the present invention may direct the healthcare provider to supplement the interaction with an artificial reality device, such as an augmented reality (AR) system or a virtual reality (VR) system. In various embodiments, the system may determine that the AR system is to be used. In various embodiments, the system may determine that the VR system is to be used. The AR system and/or the VR system may be used to demonstrate a surgical procedure or other medical procedure to the patient to facilitate a better understanding of the procedure. The system may retrieve the patient's medical record from, for example, an electronic health/medical record (EHR/EMR database) as is known in the art, including one or more medical images (e.g., x-ray, CT, MM, and/or ultrasound) corresponding to an anatomical structure of the patient (e.g., a bone, an organ, muscle, tendon, ligament and/or other anatomical structure).

The system may generate from the one or more medical images, a three-dimensional simulation of the patient's anatomy in the artificial reality device (e.g., the AR system and/or the VR system). Once the three-dimensional simulation of the patient's anatomy is generated, the system may generate a three-dimensional (3D) simulation of a surgical/medical procedure (e.g., hip/knee replacement, cardiac bypass surgery, stent placement, tumor removal, spinal fusion, biopsy procedure, or diagnostic procedure) in the artificial reality device (e.g., the AR system and/or the VR system) to be viewed by the patient with the healthcare provider. During or after the 3D simulation, the healthcare provider may guide the patient through a more detailed explanation of the surgical/medical procedure or treatment. In various embodiments, the three-dimensional simulation may include a simulation of the effects of a medical treatment plan, such as a simulation of the effect of a particular drug treatment on the patient (e.g., a simulation of statin drugs on cholesterol buildup or anti-inflammatory drug on an inflamed tissue). The system may generate one or more labels for relevant anatomical features in the three-dimensional simulation of the patient's anatomy and display the label(s) to the patient in the artificial reality device. The system may provide an interactive dialogue explaining the simulation of the surgical/medical procedure to the patient. For example, when the patient touches a particular feature of the 3D simulation, the artificial reality device provides a tailored description of the anatomical feature, condition, or surgical step to the patient. The artificial reality device may take into account demographics of the patient and provide an explanation in language that is comprehensible to the patient. During the interactive dialogue, the system may monitor for any questions asked by the patient. Upon determining that the patient has asked a question, the system may automatically pause the display of the three-dimensional simulation so that the healthcare provider may address any questions or issues that the patient has.

If the simulation of the surgical procedure is viewed in the VR system, after the simulation is complete, the system may transition to the AR system. The system may continue to use the AR system during the remainder of the healthcare communication interaction between the healthcare provider and the patient.

The system may take into account demographics of the patient and/or healthcare provider. For example, the system may use age, income, geographic location, race, ethnicity, or other suitable demographic information to aid in determining whether the patient is involved in the healthcare communication interaction.

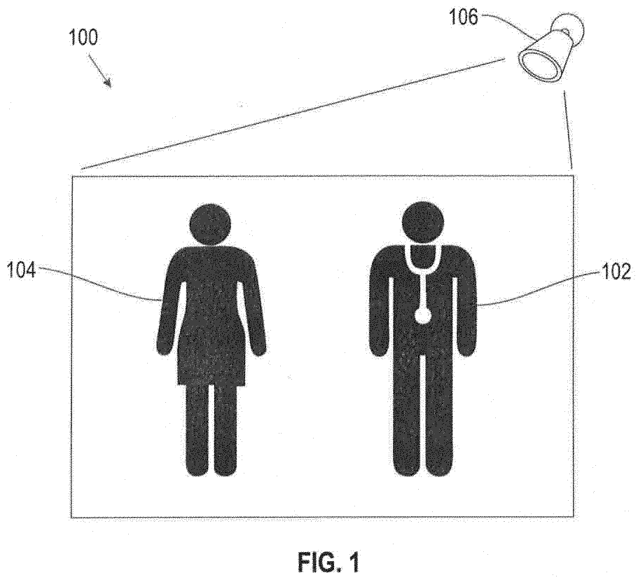

FIG. 1 illustrates a healthcare provider 102 engaging in a healthcare communication interaction with a patient 104. A system 100 includes a recording device 106 that records the communication interaction in real-time and determines a quality of the interaction based on recorded images, video, and/or audio data. For example, the system 100 may determine that the patient 104 is smiling, which may indicate that the patient 104 is relaxed and the communication interaction is positive. As another example, the system 100 may determine that the body position of the patient 104 is square and facing the healthcare provider 102, which may indicate that the patient is listening to the healthcare provider and understanding and that the communication interaction is positive.

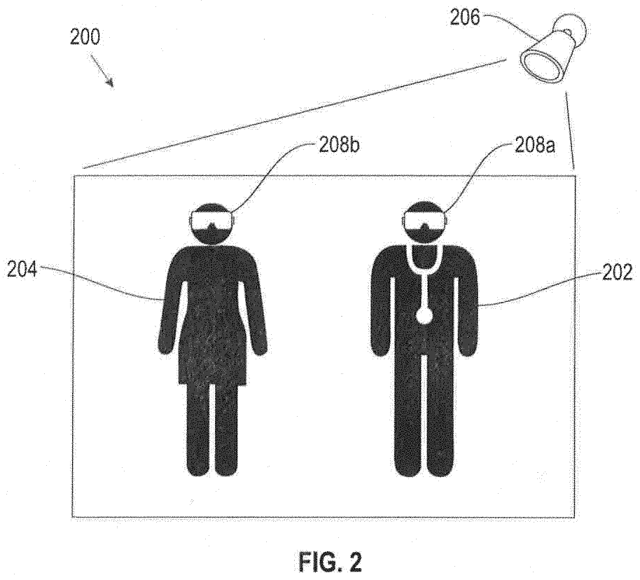

Similar to FIG. 1, the system 200 of FIG. 2 includes a recording device 206, which records a communication interaction between a healthcare provider 202 and a patient. In particular, FIG. 2 illustrates a scenario where a system 200 determines that the patient 204 may be anxious through detecting various behavioral or physiological properties, such as pacing or excessive body movement of the patient. In this scenario, the system 200 provides an indication to the healthcare provider that the communication interaction is negative and directs the healthcare provider 202 to transition to artificial reality devices, such as virtual reality devices 208a, 208b. As described in more detail above, the system 200 may retrieve medical images of the patient 204 and create a 3D simulation representing an anatomical structure of the patient 204. The healthcare provider 202 may demonstrate a surgical/medical procedure using the artificial reality devices 208a, 208b to supplement communication with the patient 204.

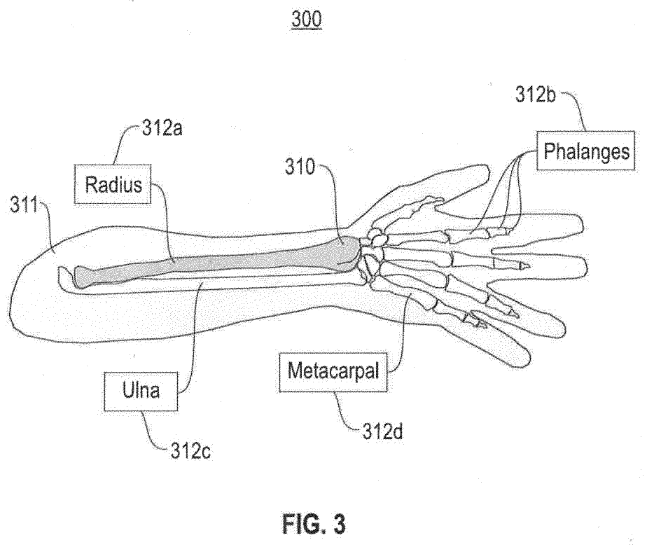

FIG. 3 illustrates an exemplary AR environment 300 where bones are simulated from patient medical images. In the example shown in FIG. 3, medical images of the bones 310 in a patient's forearm and hand are retrieved (e.g., x-rays or CT scans) from the patient's HER/EMR files using database retrieval methods as are known in the art. The system of the present invention may generate a 3D simulation of the patient's anatomy (e.g., forearm and hand bones) and displays the 3D simulation in the artificial reality device for the patient and/or the healthcare provider to view. In the AR environment 300 of the artificial reality device, the simulated bones 310 from the patient's medical images may be overlaid on a simulated arm 311 or the patient's actual arm. The AR environment 300 may generate labels 312a-312d for anatomical features of the simulation. In various embodiments, the anatomical features are interactive and may be selected/deselected by the patient to toggle the label on/off or provide additional details about the particular anatomical feature. In FIG. 3, the radius 312a is selected and is highlighted to show that it has been selected. More or fewer anatomical features may be labeled as determined by the system and/or healthcare provider. Moreover, the amount of detail on each anatomical structure may also be determined by the system and/or the healthcare provider based on, for example, patient demographics.

In various embodiments, the system of the present invention may generate a simulation of a surgical/medical procedure using the 3D simulation of the patient's anatomy. In various embodiments, the system may transition from an AR system to a VR system for the surgical/medical procedure simulation. For example, the system may generate a simulation of a carpal tunnel surgery of the wrist shown in FIG. 3 or a simulation of an open reduction and internal fixation of a broken radius/ulna (not shown).

During the 3D simulation of the surgical/medical procedure, if the patient asks a question, the system may detect that a question has been asked and automatically pause the simulation until the patient's question has been answered. The system may use data gathered from the recorded images, video, and/or audio data to determine when a question has been asked. For example, the system may determine that the patient's voice has a rising inflection, which may be indicative of a question, and pause the surgical/medical simulation. The system may resume playback of the surgical/medical procedure simulation when, for example, a voice command is provided by the healthcare provider indicating that the question has been answered. The healthcare provider may also supplement the surgical/medical procedure simulation with additional details, as needed.

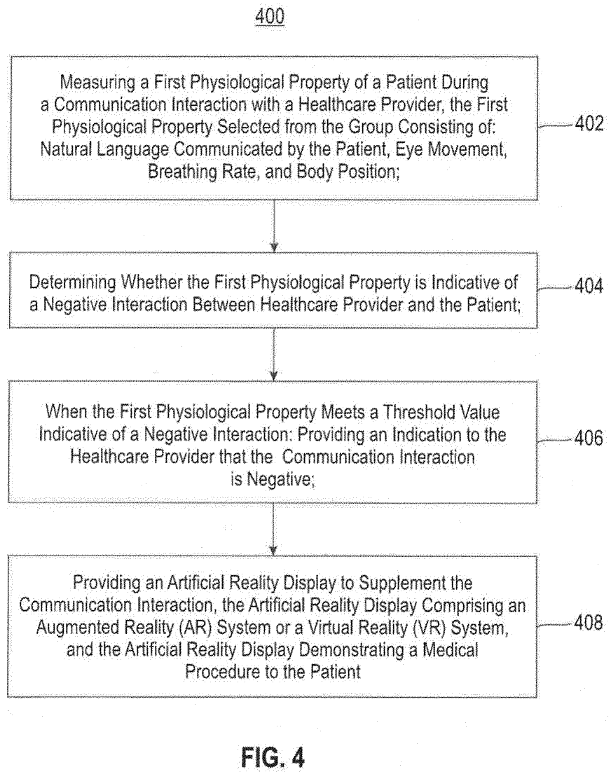

FIG. 4 illustrates a flow chart illustrating an exemplary method 400 for improving the experience between patients and healthcare providers. At 402, the method includes measuring a first physiological property of a patient during a communication interaction with a healthcare provider, where the first physiological property selected from the group consisting of: natural language communicated by the patient, eye movement, breathing rate, and body position. At 404, the method further includes determining whether the first physiological property is indicative of a negative interaction between the healthcare provider and the patient. At 406, when the first physiological property meets a threshold value indicative of a negative interaction, the method includes providing an indication to the healthcare provider that the communication interaction is negative. At 408, the method includes providing an artificial reality display to supplement the communication interaction. The artificial reality display includes an augmented reality (AR) system or a virtual reality (VR) system and demonstrates a medical procedure to the patient.

In various embodiments, a method of performing a medical procedure of the present invention may include measuring physiological properties of a patient during a communication period with a physician, the physiological properties including at least one of: natural language communicated by patient, eye movement, breathing rate, and body position. The method may further include determining whether the patient's physiological properties meet a threshold value. The method may further include providing an artificial reality display, the artificial reality display demonstrating a medical procedure to the patient when the patient's physiological properties are below the threshold value. The artificial reality may include a virtual reality or augmented reality perspective of a patient's anatomical feature, including annotations of the anatomical features depicted. Demonstration of the medical procedure may pause upon the patient asking a question. The physician may be provided at least one indicator representing the patient's understanding of the medical procedure.

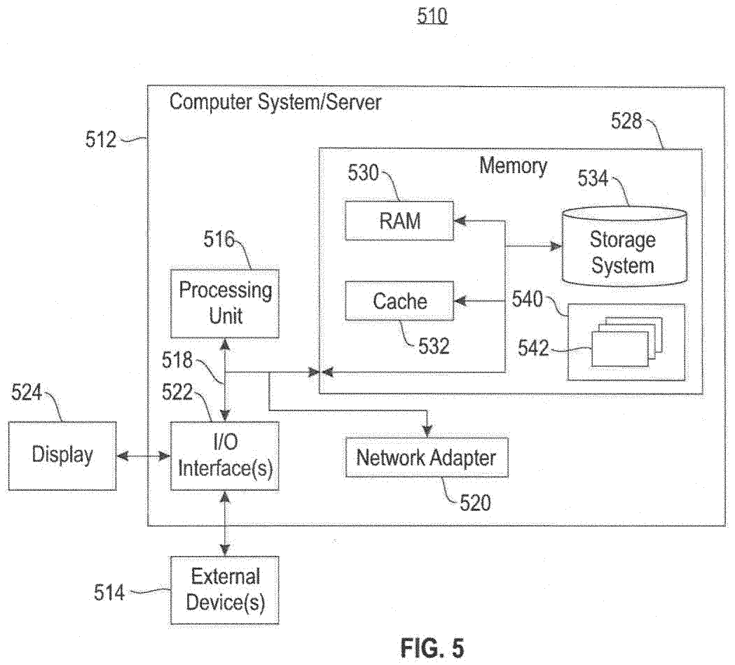

With reference to FIG. 5, a schematic of an example of a computing node is shown. Computing node 510 is only one example of a suitable computing node and is not intended to suggest any limitation as to the scope of use or functionality of embodiments of the invention described herein. Regardless, computing node 510 is capable of being implemented and/or performing any of the functionality set forth hereinabove.

In computing node 510 there is a computer system/server 512, which is operational with numerous other general purpose or special purpose computing system environments or configurations. Examples of well-known computing systems, environments, and/or configurations that may be suitable for use with computer system/server 512 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 512 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 512 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 512 in computing node 510 is shown in the form of a general-purpose computing device. The components of computer system/server 512 may include, but are not limited to, one or more processors or processing units 516, a system memory 528, and a bus 518 that couples various system components including system memory 528 to processor 516.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 512 typically includes a variety of computer system readable media. Such media may be any available media that is accessible by computer system/server 512, and it includes both volatile and non-volatile media, removable and non-removable media.

System memory 528 can include computer system readable media in the form of volatile memory, such as random access memory (RAM) 530 and/or cache memory 532. Computer system/server 512 may further include other removable/non-removable, volatile/non-volatile computer system storage media. By way of example only, storage system 534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 518 by one or more data media interfaces. As will be further depicted and described below, memory 528 may include at least one program product having a set (e.g., at least one) of program modules that are configured to carry out the functions of embodiments of the invention.

Program/utility 540, having a set (at least one) of program modules 542, may be stored in memory 528 by way of example, and not limitation, as well as an operating system, one or more application programs, other program modules, and program data. Each of the operating system, one or more application programs, other program modules, and program data or some combination thereof, may include an implementation of a networking environment. Program modules 542 generally carry out the functions and/or methodologies of embodiments of the invention as described herein.

Computer system/server 512 may also communicate with one or more external devices 514 such as a keyboard, a pointing device, a display 524, etc.; one or more devices that enable a user to interact with computer system/server 512; and/or any devices (e.g., network card, modem, etc.) that enable computer system/server 512 to communicate with one or more other computing devices. Such communication can occur via Input/Output (I/O) interfaces 522. Still yet, computer system/server 512 can communicate with one or more networks such as a local area network (LAN), a general wide area network (WAN), and/or a public network (e.g., the Internet) via network adapter 520. As depicted, network adapter 520 communicates with the other components of computer system/server 512 via bus 518. It should be understood that although not shown, other hardware and/or software components could be used in conjunction with computer system/server 512. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In various embodiments, off the shelf VR systems are optionally used with additional external compatible sensors to track various elements in multiple fields including, e.g., motion tracking, cognitive challenges, speech recognition, stability, facial expression recognition, and biofeedback.

Speech Recognition can include, but is not limited to fluent speech, ability to imitate, and pronunciation.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
measuring a first physiological property of a patient during a communication interaction with a healthcare provider, the first physiological property selected from the group consisting of: natural language communicated by the patient, eye movement, breathing rate, and body position;
determining whether the first physiological property is indicative of a negative interaction between the healthcare provider and the patient;
when the first physiological property meets a threshold value indicative of a negative interaction:
providing an indication to the healthcare provider that the communication interaction is negative;
providing an artificial reality display to supplement the communication interaction, the artificial reality display comprising an augmented reality (AR) system or a virtual reality (VR) system, and the artificial reality display demonstrating a medical procedure to the patient.

2. The method of claim 1, further comprising:
measuring a second physiological property of the healthcare provider during the communication interaction, the second physiological property selected from the group consisting of: natural language communicated by the healthcare provider, eye movement, breathing rate, and body position;
determining whether the second physiological property is indicative of a negative interaction between the healthcare provider and the patient;
when the second physiological property meets a threshold value indicative of a negative interaction, providing an indication to the healthcare provider that the communication interaction is negative.

3. The method of claim 1, further comprising:
retrieving a medical image corresponding to an anatomical structure of the patient;
generating, for display on the artificial reality display, a three-dimension simulation of the anatomical structure from the medical image.

4. The method of claim 3, further comprising:
generating, for display on the artificial reality display, a three-dimension simulation of the anatomical structure from the medical image.

5. The method of claim 4, further comprising:
labelling the anatomical structure with a plurality of labels and generating, for display in the artificial reality display, the plurality of labels.

6. The method of claim 5, further comprising providing an interactive display of the plurality of labels to the patient.

7. The method of claim 6, further comprising generating a three-dimensional simulation of a surgical procedure involving the anatomical structure in the artificial reality display.

8. The method of claim 7, further comprising:
determining whether the patient has asked a question; and
upon determining the question has been asked by the patient, pausing the three-dimensional simulation of the surgical procedure.

9. The method of claim 7, further comprising transitioning from the VR system to the AR system upon determining that the interactive dialogue is complete.

10. The method of claim 9, further comprising using the AR system until the communication interaction is complete.

11. A system comprising:
a computing node comprising a computer readable storage medium having program instructions embodied therewith, the program instructions executable by a processor of the computing node to cause the processor to perform a method comprising:
measuring a first physiological property of a patient during a communication interaction with a healthcare provider, the first physiological property selected from the group consisting of: natural language communicated by the patient, eye movement, breathing rate, and body position;
determining whether the first physiological property is indicative of a negative interaction between the healthcare provider and the patient;
when the first physiological property meets a threshold value indicative of a negative interaction:
providing an indication to the healthcare provider that the communication interaction is negative;
generating, for display on an artificial reality display, a medical procedure to the patient, the artificial reality display supplementing the communication interaction and comprising an augmented reality (AR) system or a virtual reality (VR) system.

12. The system of claim 11, further comprising a camera and a microphone for recording the communication interaction.

13. The system of claim 11, further comprising:
measuring a second physiological property of the healthcare provider during the communication interaction, the second physiological property selected from the group consisting of: natural language communicated by the healthcare provider, eye movement, breathing rate, and body position;
determining whether the second physiological property is indicative of a negative interaction between the healthcare provider and the patient;
when the second physiological property meets a threshold value indicative of a negative interaction, providing an indication to the healthcare provider that the communication interaction is negative.

14. The system of claim 11, further comprising:
retrieving a medical image corresponding to an anatomical structure of the patient;
generating, for display on the artificial reality display, a three-dimension simulation of the anatomical structure from the medical image.

15. The system of claim 14, further comprising:
generating, for display on the artificial reality display, a three-dimension simulation of the anatomical structure from the medical image.

16. The system of claim 15, further comprising:
labelling the anatomical structure with a plurality of labels and
generating, for display in the artificial reality display, the plurality of labels.

17. The system of claim 16, further comprising providing an interactive dialogue of the plurality of labels to the patient.

18. The system of claim 17, further comprising generating a three-dimensional simulation of a surgical procedure involving the anatomical structure in the artificial reality display.

19. The system of claim 17, further comprising:
determining whether the patient has asked a question;
upon determining the question has been asked by the patient, pausing the three-dimensional simulation of the surgical procedure.

20. A computer program product for performing a medical procedure, the computer program product comprising a computer readable storage medium having program instructions embodied therewith, the program instructions executable by a processor to cause the processor to:
measure a first physiological property of a patient during a communication interaction with a healthcare provider, the first physiological property selected from the group consisting of: natural language communicated by the patient, eye movement, breathing rate, and body position;
determine whether the first physiological property is indicative of a negative interaction between the healthcare provider and the patient;
when the first physiological property meets a threshold value indicative of a negative interaction:
provide an indication to the healthcare provider that the communication interaction is negative;
generating, for display on an artificial reality display, a medical procedure to the patient, the artificial reality display supplementing the communication interaction and comprising an augmented reality (AR) system or a virtual reality (VR) system.
